(19) United States
(12) Patent Application Publication
(10) Pub. No.: US 2025/0386963 A1
(43) Pub. Date: Dec. 25, 2025

(54) STRAW FOR TESTING AND PREVENTING CONSUMPTION OF AN UNSAFE BEVERAGE

(71) Applicant: Microchip Technology Incorporated, Chandler, AZ (US)

(72) Inventors: Cristina Birzan, Râmnicu Vâlcea (RO); Valentin Stoia, Bucharest (RO)

(73) Assignee: Microchip Technology Incorporated, Chandler, AZ (US)

(21) Appl. No.: 18/810,347

(22) Filed: Aug. 20, 2024

Related U.S. Application Data
(60) Provisional application No. 63/661,607, filed on Jun. 19, 2024.

(51) Int. Cl. A47G 21/18; G01N 33/14

(52) U.S. Cl. CPC A47G 21/182; G01N 33/14

(57) ABSTRACT

An apparatus and method for a straw for testing and preventing consumption of an unsafe beverage are disclosed. The apparatus may include a control circuit. The control circuit may be to instruct an electrochemical sensor to test a beverage for a harmful substance. The control circuit may also be to receive testing data from the electrochemical sensor. The control circuit may additionally be to determine whether the beverage is safe to consume and activate an electrovalve to prevent the beverage from flowing through a straw based on a determination that the beverage is unsafe to consume.

### PRIORITY

This application claims priority to U.S. Provisional Patent Application No. 63/661,607 filed Jun. 19, 2024, the contents of which are hereby incorporated in their entirety.

### TECHNICAL FIELD

The present disclosure relates to a drug detection device and, in particular, to a straw for testing and preventing consumption of an unsafe beverage.

### BACKGROUND

Drugs, such as those categorized as date rape drugs, including, for example, Rohypnol (known commonly as “roofies”), Gamma-hydroxybutyric acid (GHB), Ketamine, Ecstasy, Gamma-butyrolactone (GBL), Xanax, Valium, and Klonopin, may be used for illicit purposes to spike a person's beverage without the person's knowledge to render the person disoriented or unconscious. The presence of the drugs in a beverage may be difficult to detect by visual inspection. Additionally, personal vigilance regarding access to a person's beverage is often ineffective. Use of these drugs may increase the risk that the person drinking the spiked beverage will be the victim of a crime.

### SUMMARY OF THE INVENTION

Aspects provide systems and methods for a straw for testing and preventing consumption of an unsafe beverage. Examples of the present disclosure may include an apparatus. The apparatus may include a control circuit. The control circuit may be to instruct an electrochemical sensor to test a beverage for a harmful substance. The control circuit may also be to receive testing data from the electrochemical sensor. The control circuit may additionally be to determine whether the beverage is safe to consume and activate an electrovalve to prevent the beverage from flowing through a straw based on a determination that the beverage is unsafe to consume.

In combination with any of the above examples, the control circuit may be to receive a signal from a liquid level sensor indicating a sufficient amount of the beverage is available for testing and close the electrovalve based on the signal to prevent the beverage from flowing through the straw.

In combination with any of the above examples, the control circuit may be to open a second electrovalve and the electrovalve to allow the beverage to flow through the straw and close the electrovalve after receiving the signal from the liquid level sensor. The control circuit may also be to close the second electrovalve based on the determination that the beverage is unsafe to consume to preserve a sample of the beverage.

In combination with any of the above examples, the control circuit may be to pair with a user device and authenticate the user device. The control circuit may also be to establish a communication channel with the user device and transmit a notification to the user device that the beverage is unsafe to consume.

In combination with any of the above examples, the control circuit may be to transmit an instruction to the user device to notify a law enforcement organization that the beverage is unsafe to consume.

In combination with any of the above examples, the straw may be formed of a reusable portion including the control circuit and a disposable portion including the electrochemical sensor and the electrovalve. The control circuit may be to establish a communication channel between the reusable portion and the disposable portion.

In combination with any of the above examples, the control circuit may be to receive a command from an electrical contact and activate a second electrovalve to allow the beverage to flow through the straw.

Alone or in combination with any of the above examples, examples of the present disclosure may include a method. The method may include instructing an electrochemical sensor to test a beverage for a harmful substance. The method may also include receiving testing data from the electrochemical sensor. The method may additionally include determining whether the beverage is safe to consume and activating an electrovalve to prevent the beverage from flowing through a straw based on a determination that the beverage is unsafe to consume.

In combination with any of the above examples, the method may further include receiving a signal from a liquid level sensor indicating a sufficient amount of the beverage is available for testing and closing the electrovalve based on the signal to prevent the beverage from flowing through the straw.

In combination with any of the above examples, the method may further include opening the electrovalve and a second electrovalve to allow the beverage to flow through the straw and closing the electrovalve after receiving the signal from the liquid level sensor. The method may also include closing the second electrovalve based on the determination that the beverage is unsafe to consume to preserve a sample of the beverage.

In combination with any of the above examples, the method may further include pairing with a user device and authenticating the user device. The method may also include establishing a communication channel with the user device and transmitting a notification to the user device that the beverage is unsafe to consume.

In combination with any of the above examples, the method may further include transmitting a notification to a law enforcement organization that the beverage is unsafe to consume.

In combination with any of the above examples, the method may further include establishing a communication channel between a disposable portion of the straw containing the electrochemical sensor and the electrovalve and a reusable portion of the straw.

In combination with any of the above examples, the method may further include receiving a command from an electrical contact and activating the electrovalve to allow the beverage to flow through the straw.

Alone or in combination with any of the above examples, examples of the present disclosure may include a system including a straw. The straw may include an electrochemical sensor, an electrovalve, and a control circuit. The control circuit may be to instruct an electrochemical sensor to test a beverage for a harmful substance. The control circuit may also be to receive testing data from the electrochemical sensor. The control circuit may additionally be to determine whether the beverage is safe to consume and activate an electrovalve to prevent the beverage from flowing through a straw based on a determination that the beverage is unsafe to consume.

In combination with any of the above examples, the straw may include a liquid level sensor. The control circuit may be to receive a signal from a liquid level sensor indicating a sufficient amount of the beverage is available for testing and close the electrovalve based on the signal to prevent the beverage from flowing through the straw.

In combination with any of the above examples, the straw may include a second electrovalve. The control circuit may be to open the second electrovalve and the electrovalve to allow the beverage to flow through the straw and close the electrovalve after receiving the signal from the liquid level sensor. The control circuit may also be to close the second electrovalve based on the determination that the beverage is unsafe to consume to preserve a sample of the beverage.

In combination with any of the above examples, the system may also include a user device. The control circuit may be to pair with the user device and authenticate the user device. The control circuit may also be to establish a communication channel with the user device.

In combination with any of the above examples, the straw may be formed of a reusable portion including the control circuit and a disposable portion including the electrochemical sensor and the electrovalve. The control circuit may be to establish a communication channel between the reusable portion and the disposable portion.

In combination with any of the above examples, the straw may include an electrical contact and a second electrovalve. The control circuit may be to receive a command from an electrical contact and activate a second electrovalve to allow the beverage to flow through the straw.

### BRIEF DESCRIPTION OF THE DRAWINGS

The figures illustrate examples of systems and methods for a straw for testing and preventing consumption of an unsafe beverage.

FIG. 1 is a perspective view of a straw for detecting drugs or other harmful substances in liquid beverages, according to examples of the present disclosure;

FIG. 2 is a block diagram of a system for testing and preventing consumption of an unsafe beverage, according to examples of the present disclosure;

FIG. 3 illustrates a method performed for testing and preventing consumption of an unsafe beverage, according to examples of the present disclosure; and

FIG. 4 illustrates a more detailed method performed for testing and preventing consumption of an unsafe beverage, according to examples of the present disclosure.

The reference number for any illustrated element that appears in multiple different figures has the same meaning across the multiple figures, and the mention or discussion herein of any illustrated element in the context of any particular figure also applies to each other figure, if any, in which that same illustrated element is shown.

### DESCRIPTION

According to an aspect of the invention, a straw for testing and preventing consumption of an unsafe beverage is provided. The straw may provide non-intrusive detection of drugs or other harmful substances in a beverage and may prevent a user from consuming the beverage if the beverage is deemed unsafe. The detection may be non-intrusive to avoid aggressive behavior from people in the vicinity of the user who may be suspicious or offended by the use of the straw. For example, the straw may have the shape and appearance of a common drinking straw to not draw attention to the use of the straw for drug detection purposes. The straw may couple to another device, such as a smartphone, for issuing automated and hidden alerts to the user, and in some instances, to a law enforcement organization. The straw may provide early detection of spiked beverages by scanning the beverage as soon as the straw contacts the beverage.

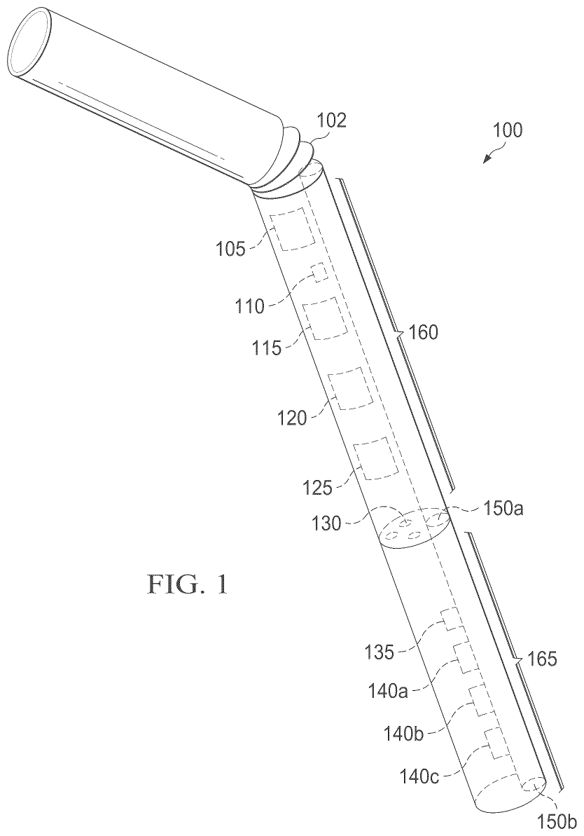

FIG. 1 is a perspective view of a straw for testing and preventing consumption of an unsafe beverage, according to examples of the present disclosure. Straw 100 may have a similar form factor as a common drinking straw. In some examples, straw 100 may include elbow 102 such that straw 100 may bend at elbow 102. In other examples, straw 100 may be curved into a shape. In some examples, straw 100 may be embedded in a beverage container. Straw 100 may include radio frequency (RF) interface 105, light-emitting diode (LED) 110, security circuit 115, control circuit 120, battery 125, one or more connection pads 130, liquid level sensor 135, one or more electrochemical sensors 140, and electrovalves 150a and 150b.

In some examples, the components of straw 100 may be divided between upper portion 160 and lower portion 165. Upper portion 160 of straw 100 may be reusable while lower portion 165 of straw 100 may be a one-time use, disposable component. In some examples, lower portion 165 of straw 100 may be reusable after cleaning the components contained therein, such as electrochemical sensors 140.

RF interface 105 may establish a connection with near-by external devices (not expressly shown in FIG. 1), such as a smartphone or wearable device, to transmit information to the device. The information transmitted to the device may include audio recordings, images, video recordings, testing results, or any combination thereof. RF interface 105 may communicate with the device using any suitable RF protocol, such as Bluetooth, near field communication (NFC), MiWi, Wi-Fi, Z-Wave, or Zigbee.

LED 110 may be used to provide notifications to a user of straw 100. For example, LED 110 may illuminate if the beverage into which straw 100 in placed is deemed unsafe. In some examples, straw 100 may omit LED 110 to allow the user of straw 100 to test a beverage more discretely.

Security circuit 115 may be used for proof of authenticity. For example, security circuit 115 may verify the compatibility of electrochemical sensors 140 with other components of straw 100. Additionally, or alternatively, security circuit 115 may be used to encrypt communications sent to an external device via RF interface 105. In some examples, lower portion 165 of straw 100 may include a security circuit (not expressly shown) to authenticate lower portion 165 of straw 100 when it is coupled to the upper portion of straw 100.

Control circuit 120 may be used to control the function of the components of straw 100. Control circuit 120 may be a central processing unit (CPU), a general purpose processor, a specific purpose processor, a microcontroller, a programmable logic controller (PLC), a digital signal processor (DSP), an application specific integrated circuit (ASIC), a field-programmable gate array (FPGA) or other programmable logic device, discrete gate or transistor logic, discrete hardware components, other programmable device, or any combination thereof designed to perform the functions disclosed herein. In some examples, straw 100 may include a real-time clock/calendar (RTCC) (not expressly shown). The RTCC may maintain accurate time, even when straw 100 is powered off. The RTCC may be used to time stamp events, such as the testing of the beverage or opening or closing of electrovalves 150.

Battery 125 may provide power to the components of straw 100. Battery 125 may be charged using one or more connection pads 130. In some examples, connection pads 130 may be electromagnetic to allow battery 125 to be charged using inductive charging. Connection pads 130 may be placed between upper portion 160 of straw 100 and lower portion 165 of straw 100. When upper portion 160 and lower portion 165 of straw 100 are uncoupled, connection pads 130 may be visible such that connection pads 130 may be coupled to a battery charging cable.

Connection pads 130 may be dual voltage and may also be used to establish a communication between the components on upper portion 160 of straw 100 (e.g., containing RF interface 105, LED 110, security circuit 115, control circuit 120, and battery 125) and the components on lower portion 165 of straw 100 (e.g., containing liquid level sensor 135, one or more electrochemical sensors 140, and electrovalves 150a and 150b). Connection pads 130 may also be used to provide power to the components of lower portion 165.

Liquid level sensor 135 may detect the level of beverage in lower portion 165. When the amount of beverage in lower portion 165 is a sufficient amount available for testing, liquid level sensor 135 may send a signal to control circuit 120 indicating that sufficient beverage is present. In response to the signal, control circuit 120 may close electrovalve 150a to stop the flow of the beverage through straw 100.

Electrochemical sensors 140 may be used to test the beverage for one or more substances such as drugs or other harmful substances. Straw 100 may include one or more electrochemical sensors 140. A given electrochemical sensor 140 may test for the presence of a given drug or harmful substance or a given electrochemical sensor 140 may test for the presence of a variety of drugs or harmful substances. For example, straw 100 may include electrochemical sensor 140a for testing for drug A, electrochemical sensor 140b for testing for drug B, and electrochemical sensor 140c for testing for drug B. As another example, straw 100 may include one electrochemical sensor 140 that tests for drug A, drug B, and drug C. In some examples, electrochemical sensor 140 may include an isolation channel to isolate the portion of the beverage tested. An isolation channel may be used in circumstances where electrochemical sensor 140 may influence the beverage during testing.

Electrovalves 150a and 150b selectively allow and disallow the beverage to flow through straw 100. For example, after straw 100 is placed in a beverage, electrovalves 150a and 150b may be opened to allow the beverage to flow through straw 100 until the beverage reaches liquid level sensor 135. When the beverage reaches liquid level sensor 135, liquid level sensor 135 may send a signal to control circuit 120 and control circuit 120 may close electrovalve 150a or 150b to prevent the beverage from flowing further upward and being consumed by the user. After the beverage has been tested, if the beverage is deemed safe for consumption, control circuit 120 may open electrovalve 150a or 150b to allow the beverage to flow through straw 100 to the user. If the beverage is deemed unsafe, electrovalve 150a or 150b may remain closed. Additionally, control circuit 120 may close the other electrovalve 150a or 150b to trap the liquid sample in lower portion 165 of straw 100 to preserve the sample for additional testing at a later time or as evidence to provide to a law enforcement organization.

In some examples, straw 100 may additionally include one or more cameras or microphones (not expressly shown) to record the surrounding environment. The camera, microphone, or any combination thereof may be activated by control circuit 120 after the beverage is deemed unsafe. For example, control circuit 120 may activate a camera to record video of the surrounding environment to capture an image of a person suspected of placing the drug or harmful substance in the beverage.

In some examples, straw 100 may additionally include an accelerometer (not expressly shown) to detect movement, rotation, or any combination thereof. Straw 100 may further include an activation circuit (not expressly shown) to allow a user to manually open electrovalve 150a, electrovalve 150b, or any combination thereof. In some examples, a user may activate electrovalve 150a, electrovalve 150b, or any combination thereof using an application on the user's device (e.g., a smartphone or wearable device).

If electrochemical sensors 140 test the beverage and determine the beverage is unsafe, control circuit 120 may communicate the test results to an external device, such as the user's smartphone or wearable device, via RF interface 105. An application executing on the external device may provide a discrete notification to the user that the beverage is unsafe (e.g., a notification on the smartphone or wearable device). Additionally, or alternatively, the application may alert a law enforcement organization. The application may communicate with the law enforcement organization via an authenticated and encrypted communication channel. The application may provide the law enforcement organization information on the circumstances of the detection of an unsafe drink, such as an identification of the user, the user's geolocation, an identification of the substances detected during testing, voice recordings, images, video recordings, or any combination thereof. In some examples, the application may alert other individuals designated by the user, such as a family member or friend of the user.

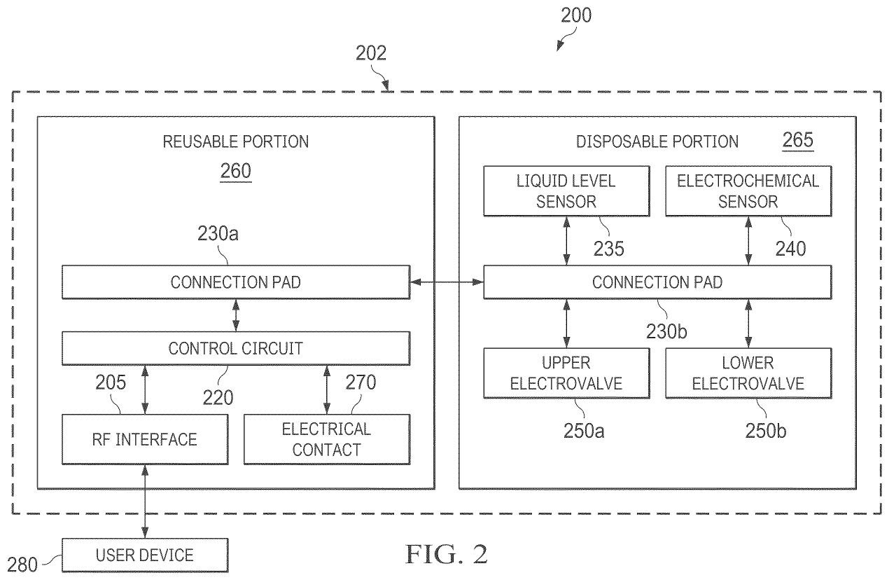

FIG. 2 is a block diagram of a system for testing and preventing consumption of an unsafe beverage, according to examples of the present disclosure. System 200 may include straw 202 and user device 280. Straw 202 may be similar to straw 100 shown in FIG. 1. Straw 202 may include RF interface 205, control circuit 220, connection pads 230a and 230b, liquid level sensor 235, electrochemical sensor 240, electrovalves 250a and 250b, and electrical contact 270. The components of straw 202 may be divided between reusable portion 260 and disposable portion 265. In some examples, disposable portion 265 may be reusable after cleaning the components contained therein, such as electrochemical sensor 240.

RF interface 205 may establish a connection with user device 280, to transmit information to user device 280. The information transmitted to the device may include audio recordings, images, video recordings, testing results, or any combination thereof. RF interface 205 may be similar to RF interface 105 shown in FIG. 1.

Control circuit 220 may be used to control the function of the components of straw 202. Control circuit 220 may be similar to control circuit 120 shown in FIG. 1.

Connection pads 230a and 230b may facilitate communication between reusable portion 260 and disposable portion 265 and provide power to the components of disposable portion 265. Connection pads 230a and 230b may be similar to connection pads 130 shown in FIG. 1. Connection pad 230a may be placed on reusable portion 260 and connection pad 230b may be placed on disposable portion 265.

Liquid level sensor 235 may be similar to liquid level sensor 135 shown in FIG. 1 and may detect the level of beverage in disposable portion 265. When the amount of beverage in disposable portion 265 is sufficient for testing, liquid level sensor 235 may send a signal to control circuit 220 via connection pads 230a and 230b indicating that sufficient beverage is present. In response to the signal, control circuit 220 may close upper electrovalve 250a or lower electrovalve 250b to stop the flow of the beverage through straw 202.

Electrochemical sensor 240 may be used to test the beverage for one or more substances such as drugs or other harmful substances. Electrochemical sensor 240 may be similar to electrochemical sensor 140 shown in FIG. 1.

Electrovalves 250a and 250b selectively allow and disallow the beverage to flow through straw 202. For example, after straw 202 is placed in a beverage, electrovalves 250a and 250b may be opened to allow the beverage to flow through straw 202 until the beverage reaches liquid level sensor 235. When the beverage reaches liquid level sensor 235, liquid level sensor 235 may send a signal to control circuit 220 and control circuit 220 may close upper electrovalve 250a or lower electrovalve 250b to prevent the beverage from flowing further upward and being consumed by the user. After the beverage has been tested, if the beverage is deemed safe for consumption, control circuit 220 may open upper electrovalve 250a or lower electrovalve 250b to allow the beverage to flow through straw 202 to the user. If the beverage is deemed unsafe, upper electrovalve 250a or lower electrovalve 250b may remain closed and control circuit 220 may close the other of upper electrovalve 250a or lower electrovalve 250b to trap the liquid sample in disposable portion 265 of straw 202 to preserve the sample. Electrovalves 250a and 250b may be similar to electrovalves 150a and 150b, shown in FIG. 1.

Electrical contact 270 may be an activation circuit to allow a user to manually open upper electrovalve 250a, lower electrovalve 250b, or any combination thereof. Electrical contact 270 may be a button, switch, or any other suitable mechanism used to send a signal to control circuit 220 to cause control circuit 220 to open upper electrovalve 250a, lower electrovalve 250b, or both, and allow the beverage to flow through straw 202.

If electrochemical sensors 240 test the beverage and determine the beverage is unsafe, control circuit 220 may communicate the test results to user device 280. User device 280 may be any suitable electronic device, such as a smartphone or wearable device. An application executing on user device 280 may provide a discrete notification to the user that the beverage is unsafe (e.g., a notification on user device 280). Additionally, or alternatively, the application may alert a law enforcement organization. The application may communicate with the law enforcement organization via an authenticated and encrypted communication channel. The application may provide the law enforcement organization information on the circumstances of the detection of an unsafe drink, such as an identification of the user, the user's geolocation, an identification of the substances detected during testing, voice recordings, images, video recordings, or any combination thereof. In some examples, the application may alert other individuals designated by the user, such as a family member or friend of the user.

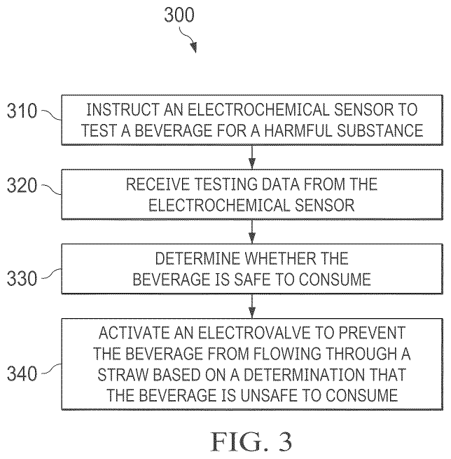

FIG. 3 illustrates a method performed for testing and preventing consumption of an unsafe beverage, according to examples of the present disclosure. Method 300 may be implemented using a control circuit, such as control circuit 120 or control circuit 220 shown in FIGS. 1 and 2, in combination with a processor, or any other system operable to implement method 300. Although examples have been described above, other variations and examples may be made from this disclosure without departing from the spirit and scope of these disclosed examples.

Method 300 may begin at block 310 where the control circuit may instruct an electrochemical sensor to test a beverage for a harmful substance. The control circuit may test the beverage by activating one or more electrochemical sensors, such as electrochemical sensors 140 or 240.

At block 320, the control circuit may receive testing data from the electrochemical sensors.

At block 330, the control circuit may determine whether the beverage is safe to consume by analyzing the testing data received at block 320. The beverage may be deemed unsafe if the presence of a drug or other harmful substance is detected.

At block 340, the control circuit may activate an electrovalve, such as electrovalve 150a, 150b, 250a, or 250b, to prevent the beverage from flowing through a straw based on a determination at block 330 that the beverage is unsafe to consume.

Although FIG. 3 discloses a particular number of operations related to method 300, method 300 may be executed with greater or fewer operations than those depicted in FIG. 3. In addition, although FIG. 3 discloses a certain order of operations to be taken with respect to method 300, the operations comprising method 300 may be completed in any suitable order.

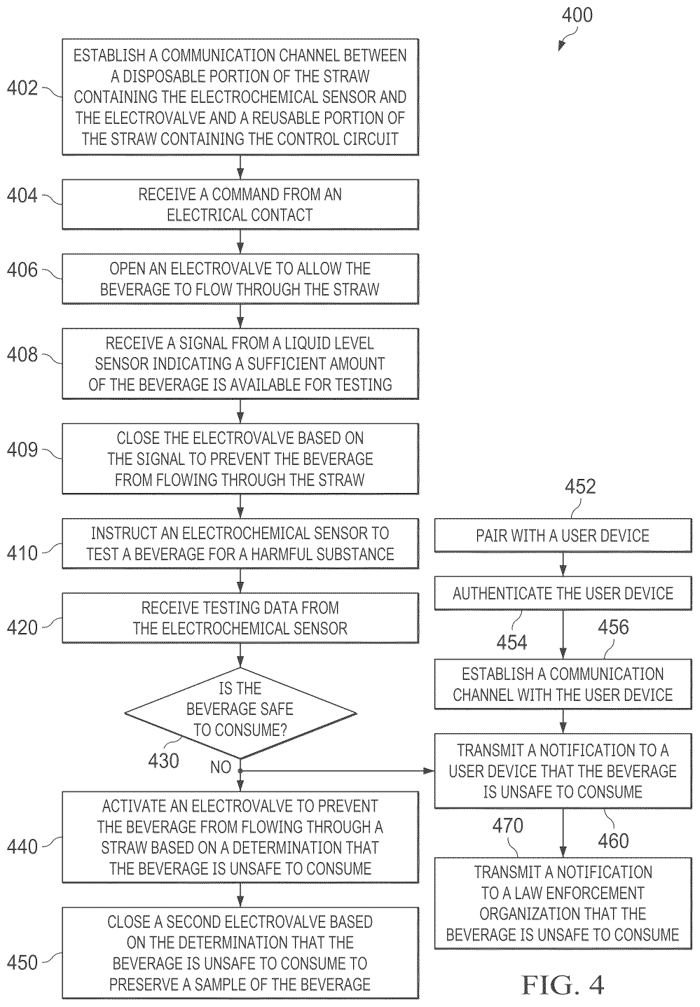

FIG. 4 illustrates a more detailed method performed for testing and preventing consumption of an unsafe beverage, according to examples of the present disclosure. Method 400 may be implemented using a control circuit, such as control circuit 120 or control circuit 220 shown in FIGS. 1 and 2, in combination with a processor, or any other system operable to implement method 400. Although examples have been described above, other variations and examples may be made from this disclosure without departing from the spirit and scope of these disclosed examples.

Method 400 may begin at block 402 where the control circuit may establish a communication channel between a disposable portion of the straw containing an electrochemical sensor and an electrovalve and a reusable portion of the straw. The communication channel may be established between connection pads on the reusable and disposable portions, such as connection pads 230a and 230b shown in FIG. 2.

At block 404, the control circuit may receive a command from an electrical contact. A user may interact with the electrical contact (e.g., press a button, activate a switch) to cause the electrical contact to send a signal to the control circuit indicating that the user has activated the electrical contact. The user may activate the electrical contact after placing the straw in a beverage to initiate testing of the beverage.

At block 406, the control circuit may open an electrovalve to allow the beverage to flow through the straw in response to the command from the electrical contact received at block 404. The control circuit may open electrovalve 150a, 150b, 250a, or 250b. In some examples, the control circuit may open the electrovalve based on the straw being placed in the beverage without any activation of the electrical contact. After the electrovalve is opened, the beverage may begin to flow through the straw.

At block 408, the control circuit may receive a signal from a liquid level sensor, such as liquid level sensor 135 or 235, indicating a sufficient amount of the beverage is available for testing. After the electrovalve is opened at block 406, the beverage may flow through the straw until it reaches the liquid level sensor, causing the liquid level sensor to send a signal to the control circuit.

At block 409, the control circuit may close the electrovalve based on the signal received at block 408 to prevent the beverage from flowing further through the straw. The beverage may remain within the lower portion of the straw.

At block 410, the control circuit may instruct an electrochemical sensor to test a beverage for a harmful substance. The control circuit may test the beverage by activating one or more electrochemical sensors, such as electrochemical sensors 140 or 240.

At block 420, the control circuit may receive testing data from the electrochemical sensors.

At block 430, the control circuit may determine whether the beverage is safe to consume by analyzing the testing data received at block 420. The beverage may be deemed unsafe if the presence of a drug or other harmful substance is detected.

At block 440, the control circuit may activate an electrovalve, such as electrovalve 150a, 150b, 250a, or 250b, to prevent the beverage from flowing through a straw based on a determination at block 430 that the beverage is unsafe to consume.

At block 450, the control circuit may close a second electrovalve, such as electrovalve 150a, 150b, 250a, or 250b, to preserve a sample of the beverage within the disposable portion of the straw. The sample may be preserved for additional testing or as evidence for a law enforcement organization.

At block 452, the control circuit may pair the straw with a user device, such as a smartphone or wearable device. The pairing process may occur over an RF link, such as using an RF interface, such as RF interface 105 or 205 shown in FIGS. 1 and 2.

At block 454, the control circuit may authenticate the user device. The control circuit may perform a mutual authentication with an application executing on the user device.

At block 456, after authentication at block 454, the control circuit may establish a communication channel for communications between the straw and the user device. The communication channel may be secure (e.g., encrypted). The application executing on the user device may run in the background and may receive a live data feed from the straw. In some examples, the application may be a hidden application or present as a fake application.

At block 460, the control circuit may transmit a notification to the user device that the beverage is unsafe to consume. In some examples, the control circuit may instruct the application executing on the user device to notify the user that the beverage is unsafe.

At block 470, the control circuit may instruct the application executing on the user device to notify a law enforcement organization that the beverage is unsafe. The application may send the notification via a secure (e.g., authenticated, encrypted, or both) communications link. The application may send additional information to the authorities such as the user's identification, the chemical test results, the recordings captured of a user's environment, the user's geolocation, date and time data, or any combination thereof.

Although FIG. 4 discloses a particular number of operations related to method 400, method 400 may be executed with greater or fewer operations than those depicted in FIG. 4. In addition, although FIG. 4 discloses a certain order of operations to be taken with respect to method 400, the operations comprising method 400 may be completed in any suitable order.

Although examples have been described above, other variations and examples may be made from this disclosure without departing from the spirit and scope of these disclosed examples.

1. An apparatus, comprising:
a control circuit to:
instruct an electrochemical sensor to test a beverage for a harmful substance;
receive testing data from the electrochemical sensor;
determine whether the beverage is safe to consume; and
activate an electrovalve to prevent the beverage from flowing through a straw based on a determination that the beverage is unsafe to consume.

2. The apparatus of claim 1, wherein the control circuit is to:
receive a signal from a liquid level sensor indicating a sufficient amount of the beverage is available for testing; and
close the electrovalve based on the signal to prevent the beverage from flowing through the straw.

3. The apparatus of claim 2, wherein the control circuit is to:
open a second electrovalve and the electrovalve to allow the beverage to flow through the straw;
close the electrovalve after receiving the signal from the liquid level sensor; and
close the second electrovalve based on the determination that the beverage is unsafe to consume to preserve a sample of the beverage.

4. The apparatus of claim 1, wherein the control circuit is to:
pair with a user device;
authenticate the user device;
establish a communication channel with the user device; and
transmit a notification to the user device that the beverage is unsafe to consume.

5. The apparatus of claim 4, wherein the control circuit is to transmit an instruction to the user device to notify a law enforcement organization that the beverage is unsafe to consume.

6. The apparatus of claim 1, wherein:
the straw is formed of a reusable portion including the control circuit and a disposable portion including the electrochemical sensor and the electrovalve; and
the control circuit is to establish a communication channel between the reusable portion and the disposable portion.

7. The apparatus of claim 1, wherein the control circuit is to:
receive a command from an electrical contact; and
activate a second electrovalve to allow the beverage to flow through the straw.

8. A method, comprising:
instructing an electrochemical sensor to test a beverage for a harmful substance;
receiving testing data from the electrochemical sensor;
determining whether the beverage is safe to consume; and
activating an electrovalve to prevent the beverage from flowing through a straw based on a determination that the beverage is unsafe to consume.

9. The method of claim 8, comprising:
receiving a signal from a liquid level sensor indicating a sufficient amount of the beverage is available for testing; and
closing the electrovalve based on the signal to prevent the beverage from flowing through the straw.

10. The method of claim 9, comprising:
opening the electrovalve and a second electrovalve to allow the beverage to flow through the straw;
closing the electrovalve after receiving the signal from the liquid level sensor; and
closing the second electrovalve based on the determination that the beverage is unsafe to consume to preserve a sample of the beverage.

11. The method of claim 8, comprising:
pairing with a user device;
authenticating the user device;
establishing a communication channel with the user device; and
transmitting a notification to the user device that the beverage is unsafe to consume.

12. The method of claim 11, comprising transmitting a notification to a law enforcement organization that the beverage is unsafe to consume.

13. The method of claim 8, comprising establishing a communication channel between a disposable portion of the straw containing the electrochemical sensor and the electrovalve and a reusable portion of the straw.

14. The method of claim 8, comprising:
receiving a command from an electrical contact; and
activating the electrovalve to allow the beverage to flow through the straw.

15. A system, comprising:
a straw including:
an electrochemical sensor;
an electrovalve; and
a control circuit, the control circuit to:
instruct the electrochemical sensor to test a beverage for a harmful substance;
receive testing data from the electrochemical sensor;
determine whether the beverage is safe to consume; and
activate the electrovalve to prevent the beverage from flowing through the straw based on a determination that the beverage is unsafe to consume.

16. The system of claim 15, wherein:
the straw includes a liquid level sensor; and
the control circuit is to:
receive a signal from the liquid level sensor indicating a sufficient amount of the beverage is available for testing; and
close the electrovalve based on the signal to prevent the beverage from flowing through the straw.

17. The system of claim 16, wherein:
the straw includes a second electrovalve; and
the control circuit is to:
open the second electrovalve and the electrovalve to allow the beverage to flow through the straw;
close the electrovalve after receiving the signal from the liquid level sensor; and
close the second electrovalve based on the determination that the beverage is unsafe to consume to preserve a sample of the beverage.

18. The system of claim 15, comprising:
a user device; and
the control circuit is to:
pair with the user device;
authenticate the user device; and
establish a communication channel with the user device.

19. The system of claim 15, wherein:
the straw is formed of a reusable portion including the control circuit and a disposable portion including the electrochemical sensor and the electrovalve; and
the control circuit is to establish a communication channel between the reusable portion and the disposable portion.

20. The system of claim 15, wherein:
the straw includes an electrical contact and a second electrovalve; and
the control circuit is to:
receive a command from the electrical contact; and
activate the second electrovalve to allow the beverage to flow through the straw.
